(19) United States
(12) Patent Application Publication
(10) Pub. No.: US 2007/0038097 A1
(43) Pub. Date: Feb. 15, 2007

(54) Introducer

(76) Inventors: Alan D. Crawford, Burbank, CA (US); Spencer L. MacKay, Agoura Hills, CA (US); James J. Elist, Beverly Hills, CA (US); Warren S. Grundfest, Los Angeles, CA (US); David J. Rubenstein, Woodland Hills, CA (US); Harold B. Rose, Tarzana, CA (US); Robert M. Woskow, Encino, CA (US)

Correspondence Address: COOLEY GODWARD KRONISH LLP;ATTN: PATENT GROUP, THE BOWEN BUILDING, 875 15TH STREET, N.W. SUITE 800, WASHINGTON, DC, 20005-2221, US

(21) Appl. No.: 11/192,464

(22) Filed: Jul. 22, 2005

(51) Int. Cl. A61B 8/00

(52) U.S. Cl. 600/439

(57) ABSTRACT

An introducer for use with an ultrasonic imaging system. The introducer comprises a tubular member having a connecting end, a positioning end, and a central lumen, the connecting end including a coupling for attaching the introducer to an ultrasonic imaging system, and the positioning end comprising a plug or other structure that facilitates positioning of the introducer inside a urethra at a fixed anatomical landmark.

### FIELD OF THE INVENTION

The present invention relates to ultrasonic imaging systems and, in particular, to an introducer for use with an ultrasonic imaging system. In a particularly preferred embodiment, the invention relates to an introducer for use with transurethral ultrasonic imaging systems.

### BACKGROUND OF THE INVENTION

When performing ultrasonic imaging within a tissue region or body lumen, such as a patient's urethra, it is important to ensure proper coupling of the energy generated and received by an imaging transducer to the surrounding tissues, and it is also often important to protect the surrounding tissues from abrasion or other harm.

The present invention is directed to an introducer that functions to enhance energy transfer or acoustic coupling between an imaging transducer that is placed within a body lumen, such as a patient's urethra, and surrounding tissues. In various embodiments, the present invention also relates to the configuration, storage, and packaging of such an introducer.

### SUMMARY OF THE INVENTION

Exemplary embodiments of the present invention that are shown in the drawings are summarized below. These and other embodiments are more fully described in the Detailed Description section. It is to be understood, however, that there is no intention to limit the invention to the forms described in this Summary of the Invention or in the Detailed Description. One skilled in the art can recognize that there are numerous modifications, equivalents and alternative constructions that fall within the spirit and scope of the invention as expressed in the claims.

In one presently preferred embodiment, an introducer is provided which comprises a tubular member defining a lumen wherein the tubular member comprises an open proximal end that is adapted to connect to an ultrasonic imaging system to allow insertion of an ultrasonic imaging catheter into the lumen. The tubular member further comprises a closed distal end and an imaging section that is transparent to ultrasonic waves. The introducer also may contain an acoustic coupling medium to enhance acoustic coupling between the ultrasonic imaging catheter, the tubular member, and any surrounding tissues. A seal may be used to retain the acoustic coupling medium within the lumen, and in such embodiments, the seal preferably is breachable or may be penetrated by the ultrasonic imaging catheter.

In the above described embodiment, the seal may be provided, for example, by a cap structure, a releasable tubular seal, or a clamped fold of the tubular member. And, depending upon the intended application, the acoustic coupling medium may comprise water, some other type of liquid, such as saline solution, or a gel.

In still further embodiments, the introducer may include a distal tip or plug that may be used to position the distal end of the introducer within a particular region of interest, such as adjacent a patient's bladder sphincter or bladder neck.

As previously stated, the above-described embodiments and implementations are for illustration purposes only. Numerous other embodiments, implementations, and details of the invention are easily recognized by those of skill in the art from the following descriptions and claims.

### BRIEF DESCRIPTION OF THE DRAWINGS

FIG. 1A illustrates a sectional view of an introducer, in accordance with an embodiment of the invention. FIG. 1B illustrates the introducer shown in FIG. 1A connected to an ultrasonic imaging system.

FIG. 2 is an illustration of an introducer, covered with a sheath, and including a fold-over clamp seal, prior to the introducer's insertion into a patient's urethra, in accordance with an embodiment of the invention.

FIG. 3A and FIG. 3B illustrate an introducer system with a hydrophilic coating, in accordance with an embodiment of the invention. FIG. 3A shows the introducer before a catheter has been inserted into the lumen. FIG. 3B shows the introducer with the catheter in the process of being inserted.

FIG. 4 is a sectional schematic anatomical view showing an ultrasonic imaging catheter within an introducer inside a patient's prostatic urethra, in accordance with an embodiment of the invention.

### DETAILED DESCRIPTION

For the sake of convenience, several terms used to describe various human anatomical structures and embodiments of the invention are defined below. It should be understood that these are provided merely to aid the understanding of the description, and that the definitions should in no way limit the scope of the invention, which is defined by the appended claims.

Anterior: Situated at the front or the front surface of an organ.

Apex of the prostate: The end of the prostate gland located farthest away from the urinary bladder.

Axial/Longitudinal: Along the centerline of the urethra, regardless of patient position.

Biopsy: The removal of a small sample or samples of tissue for examination under a microscope or other device.

Bladder: The hollow organ that stores and discharges urine from the body.

Bladder neck: The outlet area of the bladder. It is composed of circular muscle fibers (bladder sphincter), and helps control urine flow from the bladder into the urethra.

Catheter drive mechanism: A motion control system that can provide axial and/or rotational motion to an imaging catheter, or an ultrasonic transducer disposed within an imaging catheter.

Distal: Remote, farther from any point of reference (the opposite of proximal).

Genitourinary system: Pertaining to the genital and urinary systems.

Imaging catheter: A tubular mechanism, containing an ultrasonic transducer for organ-tissue imaging.

Inferior: Anatomically refers to a lower surface of an organ, or a location situated below a given reference point.

Intraluminal: Within a lumen, such as a vessel or other tubular passage within the body, an organ of the body, or an area of tissue within the body.

Introducer: A device that facilitates the insertion of a catheter into the urethra.

Periluminal: Around a lumen, such as a vessel or other tubular passage within the body, an organ of the body, or an area of tissue within the body.

Posterior: Situated at the back or the back surface of an organ.

Prostatic Urethra: The segment of the urethra, which is surrounded by prostatic tissue from the proximal end at the bladder neck to the distal end at the apex of the prostate gland.

Proximal: Closer to any point of reference.

Superior: Anatomically refers to an upper surface of an organ, or situated above a given reference point.

Transducer: A device, which transforms one form of energy to another form of energy (e.g. electrical to acoustical energy, or, conversely, acoustical to electrical energy).

Transurethral: A procedure performed through the urethra.

Transverse: Placed crosswise, situated at right angles to the long axis of an organ.

Various embodiments of the invention are directed to an introducer for use with a transurethral ultrasonic imaging system. Some embodiments include an acoustic coupling medium, and others include a sheath which covers the introducer, protecting it from contamination. A further embodiment includes a sheath and a lubricant to facilitate insertion of the introducer into a patient. Other embodiments include a hydrophilic coating on the introducer to enhance lubricity and elements to facilitate hydrating the hydrophilic coating. Some embodiments have a rigid plug at the tip of the introducer to facilitate proper placement of the ultrasonic transducer prior to imaging. The system elements and various embodiments of the invention are described in detail with reference to the appended drawings.

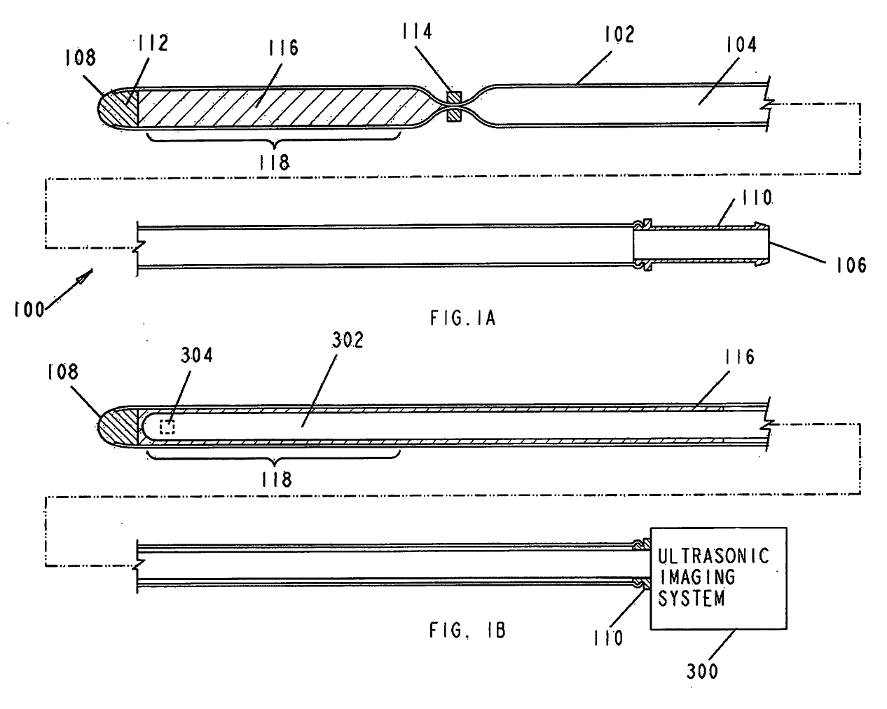

FIG. 1A illustrates a sectional view of an introducer 100 in accordance with an embodiment of the invention. Introducer 100 includes tubular member 102 which provides the primary structure of the introducer 100. Lumen 104 is the passageway or opening within the tubular member 102. The proximal end 106 of the introducer 100/tubular member 102 is open. The distal end 108 of the introducer 100/tubular member 102 is closed.

At the proximal end 106 the introducer 100 has a connector 110. At the distal end 108 there is a distal seal 112 which performs the function of closing the distal end 108 to the passage of fluid. FIG IA illustrates the distal seal as a plug. In another embodiment (not shown) the distal seal is formed from the end of the tubular member 102 without additional material.

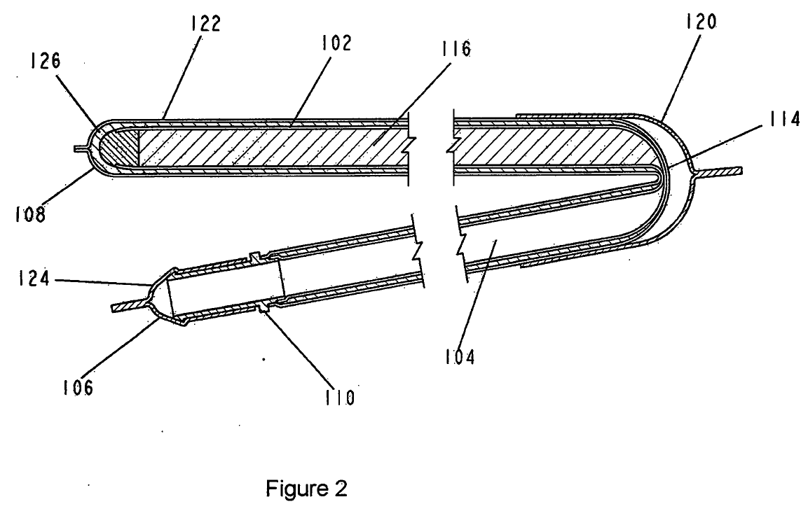

At another point along the length of the tubular member 102 a proximal seal 114 is situated. FIG. 1A illustrates the proximal seal 114 as a removable clamp. In another embodiment (see FIG. 2) the proximal seal 114 is created by folding the tubular member 102 over on itself and securing it in that position until it is ready to be used.

Within the tubular member 102 and between the distal seal 112 and the proximal seal 114 a volume is defined. This volume is filled with an acoustic coupling medium 116. The purpose of acoustic coupling medium is to allow passage of ultrasonic waves with minimal loss. Air is a very poor coupling medium for ultrasonic imaging. Examples of good ultrasonic coupling media are: pure water, water with certain additives, certain mineral oils, and certain gels.

FIG. 1B illustrates the introducer 100 shown in FIG. 1A ready for use. The proximal seal 114 has been removed. An ultrasonic imaging catheter 302, which is part of an ultrasonic imaging system 300, has been inserted in through the lumen 104 of the introducer 100. Introducer 100 has been connected to the ultrasonic imaging system using the connector 110.

The tip 306 of the ultrasonic imaging catheter 302 contains an ultrasonic transducer 304 whose purpose is to send out ultrasonic waves and detect the reflections of those waves from non-homogeneous regions of the organ being imaged. The ultrasonic imaging system 200 may rotate the ultrasonic transducer 304 to obtain planar or conical image slices. The ultrasonic imaging system 300 may also move the transducer 304 axially to obtain a 3-D image.

In order for the ultrasonic imaging system 300 to function properly the ultrasonic waves emanating from and returning to the transducer 304 should not be impeded. There is therefore an imaging section 118 of the introducer, corresponding to the axial travel of the transducer 304, where the tip of the catheter 306 should remain immersed in the coupling medium 116 and the tubular member 102 should be transparent to ultrasonic waves. An example of a structural material that is transparent to ultrasonic waves is polyethylene.

In accordance with an embodiment of the invention the tubular member 102 has an outside diameter of approximately 4 mm. The outside diameters of other embodiments fall in the range of 2.5 mm to 8 mm. In accordance with an embodiment of the invention the wall thickness of the tubular member 102 is approximately 0.2 mm. The wall thickness of other embodiments fall in the range of 0.1 mm to 0.5 mm

Turning now to FIG. 2, in accordance with an embodiment of the invention an introducer is shown as packaged prior to use. A closed-ended sheath 122 covers a tubular member 102 of the introducer from a distal end 108 to a connector 110 at a proximal end 106. This sheath 122 helps maintain cleanliness and sterility of the tubular member 102 during handling, as an imaging catheter is being inserted into the introducer, and as the introducer is connected to an ultrasonic imaging system. The sheath 122 is removed prior to the tubular member 102 being inserted into a patient's urethra.

In accordance with an embodiment of the invention a proximal seal 114 is created by folding the tubular member 102 over on itself. Those skilled in the art will note that this construction is valid with or without the sheath 122. A restraint 120 maintains the seal until the restraint 120 is removed by the user in order to insert an imaging catheter into the catheter.

In accordance with another embodiment of the invention, a cap 124 covers the open proximal end 106 of the introducer. The cap 124 is attached to the connector 1 10. The cap 124 is removed prior to the insertion of an imaging catheter and prior to the connection of the introducer to an ultrasonic imaging system.

In accordance with an embodiment of the invention a lubricant 126 occupies the space between the outside of the tubular member 102 and the inside of the sheath 122. As the sheath 122 is removed a substantial amount of lubricant 126 remains with the tubular member 102. This lubricant aids in the safe insertion of the tubular member 102 into the patient's urethra.

The sheath 122 may be constructed of a flexible, impermeable material such as polyethylene.

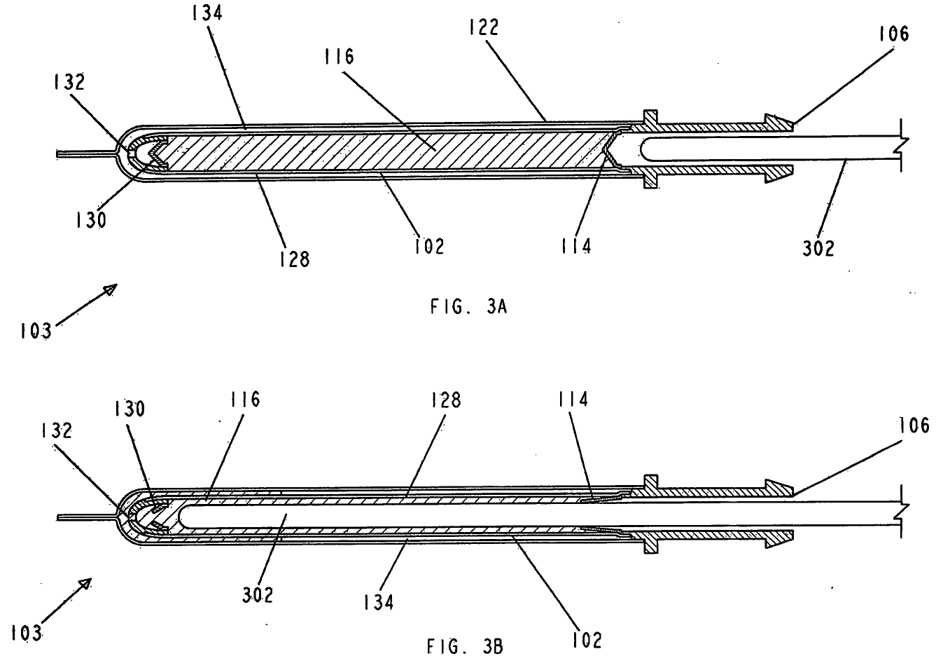

FIG. 3A and FIG. 3B, in accordance with an embodiment of the invention, illustrate a hydrophilic introducer system 103. The hydrophilic introducer system 103 includes a tubular member 102 which has a hydrophilic coating 128 on its outer surface. The purpose of the hydrophilic coating is to reduce friction between the tubular member 102 and the patient's tissue as the tubular member 102 is inserted into the patient's urethra. An example of a hydrophilic coating is a hyaluronan-based coating. Hydrophilic coatings should be immersed in water, typically for 30 seconds, before they are ready for use.

The hydrophilic introducer system 103 also may include a proximal seal 114, a water-based acoustic coupling medium 116, a sheath 122, a distal one-way valve 130, and a distal opening 132.

FIG. 3A shows the hydrophilic introducer system 103 before a catheter 302 has been fully inserted. The coupling medium 116 is constrained within the tubular member 102 between the proximal seal 114 and the distal one-way valve 130. During normal handling of the introducer system the coupling medium 116 will remain so constrained and the annular space 134 between the tubular member 102 and the sheath 122 will remain dry.

FIG. 3B shows the hydrophilic introducer system 103 with the catheter 302 in the process of being inserted in through the tubular member 102. The catheter 302 has passed the proximal seal 114 which now seals against the catheter 302 to prevent the coupling medium 116 from leaking out the proximal end 106. As the catheter 302 displaces the coupling medium 116 within the tubular member 102 the coupling medium 116 is forced past the one-way valve 130, through the distal opening 132 and into the annular space 134 where it comes in contact with the hydrophilic coating 128. The arrows in the figure represent movement of the coupling medium 116.

In another embodiment of the invention the proximal seal 114 also acts as a one-way valve—allowing air from outside to enter as the catheter 302 is being withdrawn.

In another embodiment of the invention there is a proximal one-way valve (not shown) in addition to the proximal seal 114.

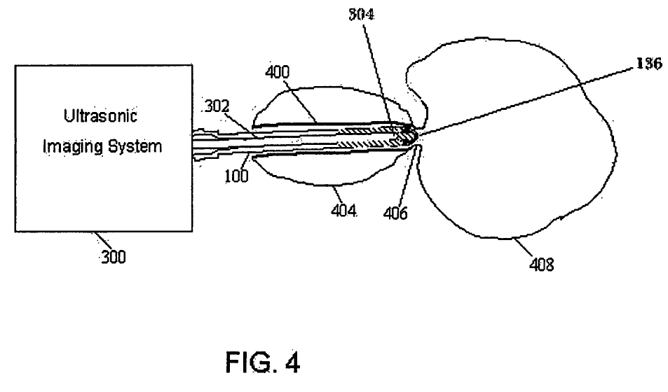

Turning now to FIG. 4, in accordance with an embodiment of the invention, a sectional anatomical view showing the ultrasonic imaging catheter 302 within the introducer 100 and inside a patient's prostatic urethra 400 is illustrated. The ultrasonic imaging catheter 302 is connected to the ultrasonic imaging system 300 for imaging the patient's prostate gland 404. In accordance with an embodiment of the invention, the ultrasonic imaging system 300 is a transurethral prostate imaging system. In accordance with another embodiment of the invention, the ultrasonic imaging catheter 302 is connected to a drive mechanism of the ultrasonic imaging system 300 (not shown in FIG. 4). The drive mechanism of the ultrasonic imaging system 300 rotates an ultrasonic transducer 304 inside the ultrasonic imaging catheter 302. In accordance with another embodiment of the invention the drive mechanism of the ultrasonic imaging system 300 can also move the imaging catheter 302 axially relative to the introducer 100 enabling the ultrasonic imaging system 300 or its adjuncts to construct a three dimensional image of the prostate.

During a 3-D imaging session it is important to place the ultrasonic transducer 304 in a suitable position before imaging begins due to mechanical travel limitations and patient discomfort considerations. For example, if a complete scan of the prostate 404 is desired, using an ultrasonic imaging system 300 which withdraws the imaging catheter 302 while imaging, then the transducer 304 should initially be positioned (within the urethra) so the initial 2-D image is on the distal side of, yet close to, the prostate 404, the distal side of the prostate 404 being the side towards the urinary bladder 408.

In accordance with an embodiment of the invention the introducer 100 includes a relatively rigid plug 136 at the distal tip. In other embodiments this rigid structure may assume other configurations, such as a thickened or enlarged section of the tubular member 102. The purpose of the rigid plug 136 is to define the distal tip of the catheter 100 as it is being inserted into the urethra. When the rigid plug 136 reaches the bladder sphincter 406 there will be increased resistance when inserting the introducer 100. This is a signal for the person inserting the introducer 100 to stop as this point closely approximates the ideal starting point for the ultrasonic transducer 304. During imaging the introducer 100 remains stationary relative to the patient while axial movement of the imaging catheter 302 occurs relative to the introducer 100.

It will be evident to a person ordinarily skilled in the art that one or more of the embodiments mentioned above provide the following advantages during transurethral ultrasonic imaging of the genitourinary system of a patient. The introducer may significantly reduce the incidence of cross-contamination between patients since it isolates the ultrasonic imaging catheter from contact with the patient's bodily fluids, and since it is disposable due to its relatively low manufacturing cost. The introducer may serve as an anchoring device, maintaining a fixed relationship to the patient as the ultrasonic imaging catheter is moved axially, thus allowing precise control of the movement of the ultrasonic transducer relative to the patient's organ being imaged. A rigid plug at the tip of the introducer may aid in the proper initial placement of the ultrasonic transducer. The sheath helps maintain cleanliness and sterility of the introducer even after the introducer is installed on the ultrasonic imaging system and up to the point the introducer is inserted into a patient's urethra. The sheath may contain a lubricant to facilitate insertion of the introducer into a patient's urethra. The introducer may have a hydrophilic coating to provide lubricity. A sheath and associated valves may enable convenient wetting of the hydrophilic coating.

While the invention is susceptible to various modifications and alternative forms, specific examples thereof have been shown by way of example in the drawings and are herein described in detail. It should be understood, however, that the invention is not to be limited to the particular forms or systems or methods disclosed, but to the contrary, the invention is to cover all modifications, equivalents, and alternatives falling within the spirit and scope of the appended claims.

What is claimed is:

1. An introducer for use with a transurethral ultrasonic imaging system, the introducer comprising:
a tubular member having a connecting end, a positioning end, and a central lumen, the connecting end including a coupling for attaching the introducer to an ultrasonic imaging system, and the positioning end comprising a plug that facilitates positioning of the introducer inside a urethra at a fixed anatomical landmark.

2. The introducer of claim 1 wherein the central lumen comprises a proximal seal and a distal seal.

3. The introducer of claim 2 wherein the proximal seal and the distal seal are one-way valves.

4. The introducer of claim 2 wherein a liquid is disposed between the proximal seal and the distal seal provides an acoustic coupling to an ultrasonic transducer of the acoustic imaging catheter.

5. The device of claim 1 wherein the fixed anatomical landmark comprises a neck region of a patient's urinary bladder.

6. The introducer of claim 1 wherein the introducer further comprises a sheath for covering and protecting the introducer prior to use.

7. The introducer of claim 1 wherein the introducer is made of a biocompatible polyolefin material.

8. The introducer of claim 1 wherein the introducer has an outer wall comprising a hydrophilic material that facilitates insertion of the introducer into a urethra.

9. The introducer of claim 1, wherein the central lumen includes a releasable seal.

10. The introducer of claim 9 wherein the releasable seal is formed by compressing the tubular member using a removable clamp.

11. The introducer of claim 9 wherein the releasable seal is created by restraining the tubular member in a folded-over configuration and breaching of the seal is accomplished by unfolding the tubular member and inserting the ultrasonic imaging catheter into the lumen.

12. An introducer for use with a prostatic imaging system, the introducer comprising:
a tubular member having a connecting end, a positioning end, and a central lumen for receiving an imaging catheter,
the connecting end having a coupling for fixing a relationship between the introducer and a drive mechanism of the prostatic imaging system,
the positioning end comprising a plug for aiding in positioning the imaging catheter within a prostatic urethra in a region adjacent to a neck of a urinary bladder and providing a reference point during transurethral imaging of a prostatic region, and
the central lumen comprising a proximal seal adjacent to the connecting end and a distal seal adjacent to the positioning end.

13. An introducer for use with a prostatic imaging system, the introducer comprising:
a tubular member having a central lumen configured to receive an imaging catheter, the central lumen having a distal imaging section through which ultrasonic imaging can be conducted and an imaging catheter can be axially displaced, the distal imaging section having a distal seal and a proximal seal and having an ultrasonic imaging coupling medium disposed between the distal and the proximal seal.

14. The introducer of claim 13, wherein the ultrasonic imaging coupling medium is selected from a group consisting of liquid and gelatinous substances.

15. The introducer of claim 13, wherein the proximal seal is formed by an external clamping mechanism that may be removed during use.

16. The introducer of claim 13, wherein the proximal seal is formed by a folding a section of the central lumen, the section can be unfolded prior to use.

17. The introducer of claim 13, wherein the distal seal comprises a plug.

18. The introducer of claim 13, wherein the distal seal comprises a closed tip of the central lumen.

19. A packaging system for an introducer comprising a tubular member defining a lumen, the tubular member comprising an open proximal end adapted to connect to an ultrasonic imaging system and adapted to allow insertion of an ultrasonic imaging catheter into the lumen, the tubular member further comprising a distal end, the packaging system comprising:
a removable closed-ended sheath adapted to cover the introducer from the distal end up to the proximal end without impeding the insertion of the ultrasonic imaging catheter or the connection of the ultrasonic imaging system, the packaging system further comprising a removable barrier adapted to cover the open proximal end of the introducer.

20. The packaging system of claim 19 wherein the introducer further comprises a connector at the proximal end and wherein the removable barrier is a cap which fits onto the connector.

21. The packaging system of claim 19 wherein the removable barrier is a bag enclosing all of the introducer.

22. The packaging system of claim 19 further comprising a substance constrained between the outside of the tubular member and the inside of the sheath.

23. The packaging system of claim 22 wherein the substance comprises a lubricant.

24. A method of positioning an introducer for use with an ultrasonic imaging system within a selected region of a patient's urethra, the method comprising:
providing an enlarged distal tip on a distal end of the introducer, and
inserting the introducer into the urethra of the patient until the enlarged distal tip of the introducer contacts a selected anatomical landmark.

25. The method of claim 24, wherein the selected anatomical landmark comprises a bladder sphincter.
